(19) United States
(12) Patent Application Publication
(10) Pub. No.: US 2009/0088801 A1
(43) Pub. Date: Apr. 2, 2009

(54) SPINAL FIXATION DEVICE AND METHOD

(75) Inventor: Kevin R. Strauss, Columbia, MD (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/239,990

(22) Filed: Sep. 29, 2008

Related U.S. Application Data
(60) Provisional application No. 60/995,601, filed on Sep. 27, 2007.

(51) Int. Cl. A61B 17/70; A61F 2/44; A61B 17/04

(52) U.S. Cl. 606/246; 606/250; 623/17.16; 606/301

(57) ABSTRACT

A spinal fixation device is provided. The spinal fixation device includes first and second fixation members. Each of the fixation members include an anchor portion configured for rigid fixation to a vertebral body, an endplate member configured to engage a surface of a vertebral body, and a connecting portion connecting the anchor portion and the endplate member. The spinal fixation device further includes a spacing element configured to be received between the endplate members of the first and second fixation members

### CROSS-REFERENCE TO RELATED APPLICATIONS

The present disclosure claims the benefit of and priority to U.S. Provisional Patent Application Ser. No. 60/995,601, filed on Sep. 27, 2007, the entire contents of which are hereby incorporated by reference.

### BACKGROUND

1. Technical Field

The present invention relates to devices and methods for use in orthopedic spine surgery. In particular, the present invention relates to a device and method to maintain the space between adjacent vertebrae while providing six degrees of freedom (DOF) at that level.

2. Background Art

The human spine is comprised of thirty-three vertebrae at birth and twenty-four as a mature adult. Between each pair of vertebrae is an intervertebral disc, which maintains the space between adjacent vertebrae and acts as a cushion under compressive, bending and rotational loads and motions. A healthy intervertebral disc has a great deal of water in the nucleus pulposus, the center portion of the disc. The water content gives the nucleus a spongy quality and allows it to absorb spinal stress. Excessive pressure or injuries to the disc can cause injury to the annulus; the outer ring that holds the disc together. Generally, the annulus is the first portion of the disc that seems to be injured. These injuries are typically in the form of small tears. These tears heal by scar tissue. The scar tissue is not as strong as normal annulus tissue. Over time, as more scar tissue forms, the annulus becomes weaker. Eventually this can lead to damage of the nucleus pulposus. The nucleus begins to lose its water content due to the damage and begins to dry up. Because of water loss, the discs lose some of their ability to act as a cushion. This may lead to even more stress on the annulus and still more tears as the cycle repeats. As the nucleus loses its water content the nucleus collapses, allowing the vertebrae above and below the disc space to move closer to one another. This results in a narrowing of the disc space between the two vertebrae. As this shift occurs, the facet joints located at the back of the spine are forced to shift. This shift changes the way the facet joints work together and may cause problems in the facet joints as well.

When a disc or vertebrae is damaged due to disease or injury, standard practice is to remove part or all of the intervertebral disc, insert a natural or artificial disc spacer and construct an artificial structure to hold the affected vertebrae in place to achieve a spinal fusion. In doing so, while the diseased or injured anatomy is addressed and the accompanying pain is significantly reduced, the natural biomechanics of the spine are affected in a unique and unpredictable way and, more often than not, the patient will develop complicating spinal issues in the future.

Accordingly, there is an overall need to treat the disease or injury while maintaining or preserving the natural spine biomechanics. Normal spine anatomy, specifically intervertebral disc anatomy, allows one vertebrae to rotate with respect to its adjacent vertebrae about all three axes. Similarly, the intervertebral disc also allows adjacent vertebrae to translate along all three axes, with respect to one another.

Therefore, a need exists for an implantable device which may be used to maintain the disc space between adjacent vertebrae while allowing the spine to rotate and translate about all three axes as it does in its natural, healthy state. The implantable device may have an additional need of being introduced into the body using a posterior approach and a technique that is familiar to the surgeon. The device may also provide a pro-longed life span in the body that may withstand early implantation, as is often indicated for younger patients, and may have a limited amount of particulate debris so as to reduce complications over the useful life of the device.

### SUMMARY

Accordingly, a spinal fixation device is provided. The spinal fixation device includes a pair of fixation members. The fixation members are mirror images of one another. The fixation members each include an anchor portion, an endplate member, and a connecting portion connecting the anchor portion and the endplate member. The spinal fixation device further includes a spacing element configured to be received between the endplate members of the first and second fixation members. The connecting portions may be semi-rigid. The spacing element may be formed from a compressible material. The connection portions may be substantially “C”-shaped and the endplate members may be substantially disc-shaped. The endplate members may include a surface for engaging a vertebra. The endplate members may have another surface for engaging the spacing element.

Further provided is a method of implanting a spinal fixation device. First, a spacing element is placed between the endplate members of fixation members of a spinal fixation device. Each fixation member further includes an anchor portion and a connecting portion connecting the anchor portion to the endplate member. The endplates of the fixation members, including the spacing element, are then received within the intervertebral space. The anchor portion of the fixation members are then secured to the vertebra. The method may further include the steps of removing the facet joint prior to inserting the first and second fixation members into the vertebral space, repeating the steps to implant a second spinal fixation device within the same vertebral space.

### BRIEF DESCRIPTION OF THE DRAWINGS

The foregoing and other features of the present disclosure will become apparent to one skilled in the art to which the present disclosure relates upon consideration of the following description of the embodiments with reference to the accompanying drawings, wherein:

FIG. 1A shows an end view of an embodiment of the spinal fixation device according to the present disclosure;

FIG. 1B shows an isometric view of the spinal fixation device of FIG. 1A;

FIG. 1C shows a top view of the device of FIGS. 1A and 1B;

FIG. 2A shows an end view of the spinal fixation device of FIGS. 1A-1C after implantation in a vertebral column; and

FIG. 2B shows a top view of the spinal fixation device of FIG. 2A.

### DETAILED DESCRIPTION

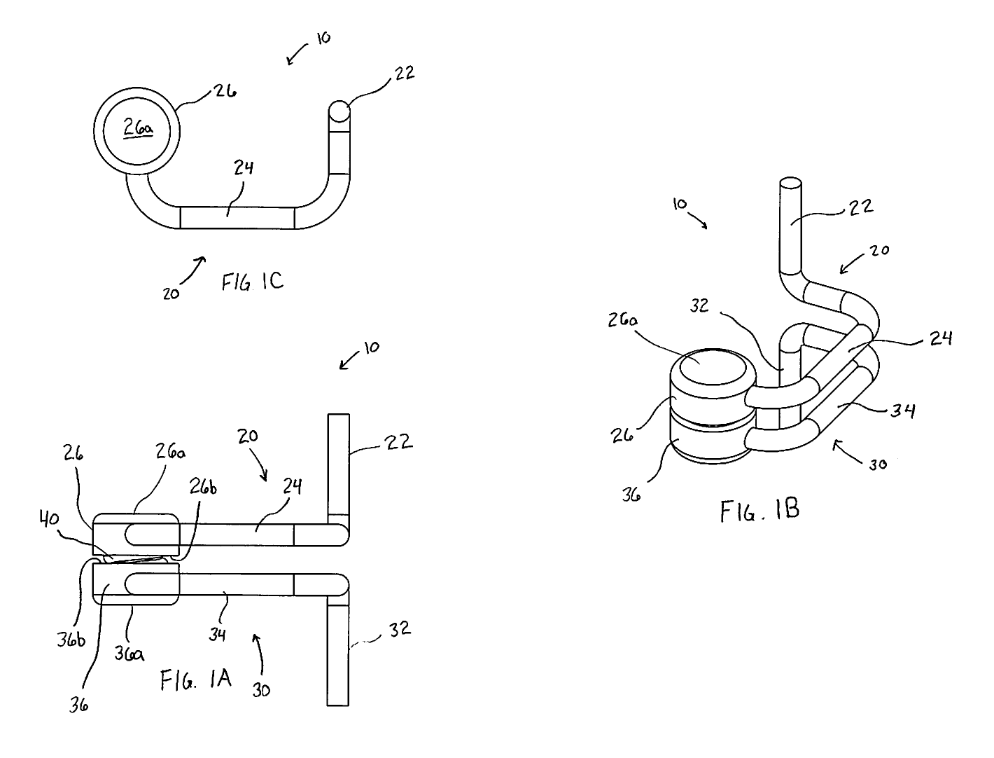

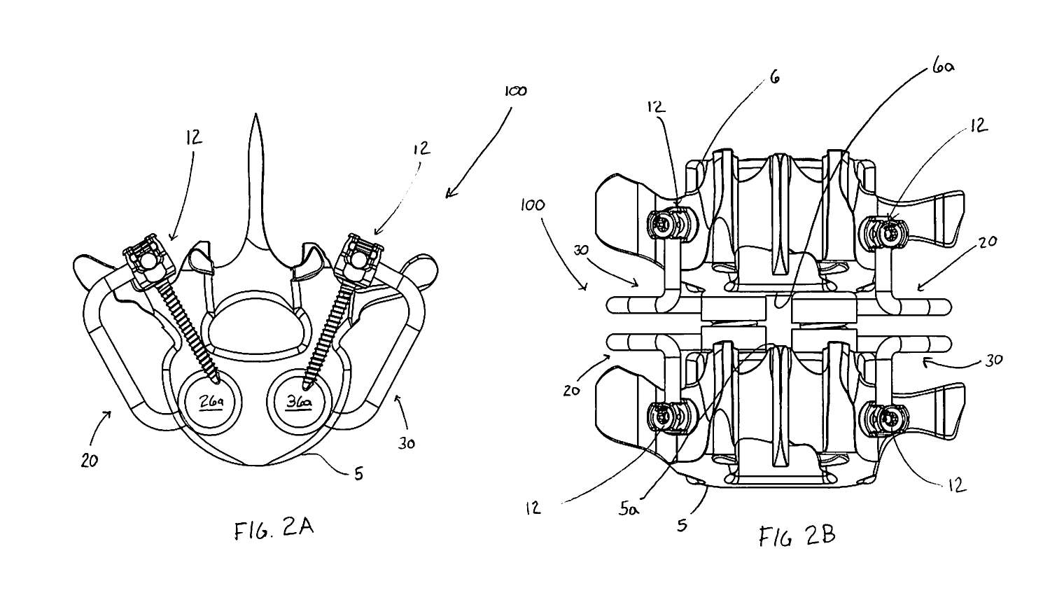

Referring initially to FIGS. 1A-1C, a spinal fixation device according to the present disclosure is shown generally as spinal fixation device 10. Spinal fixation device 10 includes first and second fixation members 20, 30 separated by a dynamic spacing element 40. As will be discussed in further detail below, spinal fixation device 10 is configured to be received between adjacent vertebrae 5, 6 (FIG. 2B) and to permit relative movement of vertebrae 5, 6 relative to each other while maintaining the spacing therebetween. In one embodiment, spinal fixation device 10 is designed to maintain the space approximately where the patient's facet joint was located.

Spinal fixation device 10 may be provided as a single unit or multiple pieces configured for assembly by a surgeon at the time of use. Spinal fixation device 10 may also be provided in a range of sizes and configurations, as will be discussed below, to better accommodate a patient's anatomy and offer greater surgical flexibility. Spinal fixation device 10 may be composed of a range of biocompatible materials including, but not limited to, titanium, titanium alloys, stainless steel, cobalt chrome and cobalt chrome alloys, ultra high molecular weight polyethylene, PEEK, and other polymers such as polycarbonate urethane. A variety of manufacturing techniques may be employed to produce spinal fixation device 10. Still referring to FIGS. 1A-1C, first and second fixation members 20, 30 are mirror images of one another and include respective bone anchoring portions 22, 32, semi-rigid connecting portions 24, 34, and endplate portions 26, 36. Bone anchoring portions 22, 32 of respective fixation members 20, 30 are configured for rigid fixation to bone anchoring members 12 (FIG. 2A). As shown, bone anchoring members 12 include pedicle screws. For a detailed discussion of the configuration, installation and operation of a compatible pedicle screw, reference is made to commonly owned U.S. Pat. No. 5,733,286 and U.S. Patent Publication 2008/0027432 which are incorporated herein by reference in their entirety. Bone anchoring members 12 may, however, include any means for securing a rod to a vertebra, including hooks, screws and other mechanical fasteners.

With reference still to FIGS. 1A-1C, semi-rigid connecting portions 24, 34 define substantially “C” shape members, although alternative configurations are envisioned. In one embodiment, fixation members 20, 30 are configured such that a surgeon may bend connection portions 24, 34 into a geometry suitable for implantation into a patient. In this manner, a jig or other device may be provided with spinal fixation device 10 to permit a surgeon to bend connection portions 24, 34 in the operating room prior to implantation. Alternatively, fixation members 20, 30 may be provided with custom shaped connecting portions 24, 34. Connecting portions 24, 34 may be integrally formed with anchor portions 22, 32 and/or endplate members 26, 36, respectively. In an alternative embodiment, connecting portions 24, 34 are securely affixed to anchor portions 22, 32 and/or endplate members 26, 36, respectively.

The semi-rigid design of connecting portion 24, 34 may serve multiple purposes, including, providing structure to spinal fixation device 10 and a means for fixating the posterior aspect of the device to the anatomy. Additionally, the inherent space between semi-rigid connection portions 24, 34 allows for translation along all three axes in the posterior aspect of the spine. This range of motion (ROM) is a significant improvement over stand-alone artificial discs and posterior dynamic stabilization devices which do not provide anterior column support. Furthermore, the inherent space between connection portion 24, 34 also allow for translation along the other two axes and rotation about all three axes, again, while continuing to provide structure and stability to the spine which was destabilized and made unbalanced due to the patient's pathology and intervening procedure.

Still referring to FIGS. 1A-1C, first and second endplate members 26, 36 include substantially disc shaped inserts including first and second surfaces 26a, 36a, 26b, 36b, respectively. First surfaces 26a, 36a of endplate members 26, 36 are configured to interface with respective surfaces 5a, 6a of vertebral bodies 5, 6 (FIG. 2B), respectively. First surfaces 26a, 36a may include texturing or other passive fixations means, i.e. spikes or keels, (not shown) for selectively engaging respective vertebral bodies 5, 6. Alternatively, first surfaces 26a, 36a of endplate members 26, 36, respectively, may be configured with an endplate interfacing means (not shown), configured such that endplate members 26, 36 may be articulated relative to surfaces 5a, 6a of vertebral bodies 5, 6, respectively, without subsidence or damage to vertebral bodies 5, 6, respectively. Second surface 26b, 36b of endplate members 26, 36 are configured to receive dynamic spacing element 40.

With reference still to FIGS. 1A-1C, dynamic spacing element 40 may take the form of a spring, elastic polymer member or other means that will permit rotation and translation of endplate members 26, 36 relative to each other. Dynamic spacing element 40 may be fixed to second surface 26b, 36b of respective endplate members 26, 36 such that spacing element 40 does not migrate. Alternatively, spacing element may be sufficiently captured by endplate members 26, 36 such that once implanted within a patient, spacing element 40 is less likely to disassemble.

The method for implanting spinal fixation device 10 will now be described with reference to FIGS. 2A and 2B. Although implantation of spinal fixation device 10 will be described as relates to a preferred posterior approach, it is envisioned that spinal fixation device 10 may be implanted from various approaches. Bone anchoring members 12 may be placed before or after the disc space is prepared. When bone anchoring members 12 are placed beforehand, bone anchoring members 12 may be used to assist in distracting vertebral bodies 5, 6. If the facet joint (not shown) has not already been removed, the facet joint is removed in order to gain access to the disc space. Sufficient disc material is removed to correct the pathology and accept endplate members 26, 36 and spacing element 40 of spinal fixation device 10. If bone anchoring members 10 have not yet been placed by this time in the procedure, they may now be placed. In some procedures, a surgeon may elect to implant a trial spinal fixation device (not shown) to ensure proper fit and alignment. This step is recommended, but not required. A surgeon may use this step to make any final corrections to semi-rigid connecting portions 24, 34 to ensure spinal fixation device 10 does not impinge on the exiting nerve root (not shown). Spacing element 40 is then received between endplate members 26, 36 if endplate members 26, 36 did not come with spacing element 40 already secured between the two, and endplate members 26, 36 and spacing element 40 are received with the intervertebral space. First surface 26a of first endplate member 20 engages first surface 5a of first vertebra 5 and first surface 36a of endplate member 36 engages first surface 6a of second vertebra 6. Anchoring portions 22, 32 are then secured to bone anchoring members 10. The implantation procedure is performed bilaterally to ensure proper balance and loading of the spine at the affected levels.

As discussed above, when spinal fixation device 10 is assembled and implanted within the intervertebral space, endplate members 26, 36 act as disc replacements. Dynamic spacing element 40 acts as a cushioning or shock absorbing member, similar to an intervertebral disc. Translation along all three axes is achieved either through dynamic spacing element 40 or through a combination of dynamic spacing element 40 and endplate members 26, 36 of the device articulating on first surfaces 5a, 6a of respective vertebral bodies 5, 6. In the instance when endplate members 26, 36 are intended to allow articulation, surface texturing or fixation means (not shown) are not desired and are replaced with a smooth, friction reducing surface (not shown).

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure. For example, in one embodiment, stop-plates and/or a damping element may be positioned on opposing the semi-rigid connecting portions approximately in the area where the facet joint was located. The stop-plates and/or damping element are designed to provide additional stability and limitations to the spinal fixation device's range of movement. In this manner, the spinal fixation device further approximates the normal biomechanics of the spine. In another embodiment, multiple levels of the spine may be treated using spinal fixation device. In this manner, the bone anchoring portion would have a semi-rigid connecting portion and endplate member at each end, thereby achieving fixation or articulation on both endplates of the same vertebral body. The device would still be implanted bilaterally and dynamic spacing devices would still be required to span the disc space to the next adjacent vertebra.

What is claimed is:

1. A spinal fixation device comprising:
first and second fixation members, each fixation member including:
an anchor portion configured for rigid fixation to a vertebral body,
an endplate member configured to engage a surface of a vertebral body, and
a connecting portion connecting the anchor portion and the endplate member; and
a spacing element configured to be received between the endplate members of the first and second fixation members.

2. The device of claim 1, wherein the first fixation member is a mirror image of the second fixation member.

3. The device of claim 1, wherein the connecting portions are semi-rigid.

4. The device of claim 1, wherein the spacing element is formed from a compressible material.

5. The device of claim 1, wherein the connecting portions are substantially “C”-shaped.

6. The device of claim 1, wherein the endplate members are substantially disc-shaped.

7. The device of claim 1, wherein the endplate members include a first surface for engaging a surface of a vertebral body.

8. The device of claim 7, wherein the endplate members include a second surface for engaging the spacing element.

9. The device of claim 1, wherein the anchor portions are configured for engagement with bone anchoring members.

10. The device of claim 1, wherein the anchor portion of the first fixation member is rigidly secured to a first vertebral body.

11. The device of claim 10, wherein the anchor portion of the second fixation member is rigidly secured to a second vertebral body.

12. The device of claim 11, wherein the endplate members of the first and second fixation members, including the spacing element therebetween, are received within the intervertebral space formed between the first and second vertebrae.

13. A method of implanting a spinal fixation device, the method comprising the steps of:
providing a spinal fixation device including first and second fixation members, the fixation members including:
an anchor portion configured for rigid fixation to a vertebral body;
an endplate member configured to engage a surface of a vertebral body; and
a connecting portion connecting the anchor portion and the endplate member; and
a spacing element configured to be received between the endplate members of the first and second fixation members;
placing the spacing element between the endplate members of the first and second fixation members;
inserting the first and second fixation members, including the spacing element therebetween, into a vertebral space created between a first and second vertebrae;
securing the anchor portion of the first fixation member to the first vertebra; and
securing the anchor portion of the second fixation member to the second vertebra.

14. The method of claim 13, further including the step of removing the facet joint prior to inserting the first and second fixation members into the vertebral space.

15. The method of claim 13, further including the step of repeating the steps to implant a second spinal fixation device within the same vertebral space.

16. The method of claim 13, further including the step of inserting one or more bone anchoring members into the first and second vertebrae.
